(19) United States
(12) Patent Application Publication
(10) Pub. No.: US 2007/0293765 A1
(43) Pub. Date: Dec. 20, 2007

(54) Method for Generating and Processing Images for Use in Dentistry

(76) Inventor: Marcello Marchesi, Modena (IT)

Correspondence Address: BROWDY AND NEIMARK, P.L.L.C.;624 NINTH STREET, NW, SUITE 300, WASHINGTON, DC, 20001-5303, US

(21) Appl. No.: 10/591,652

(22) PCT Filed: Oct. 4, 2004
(86) PCT No.: PCT/EP04/11066

§ 371 Date: Jul. 17, 2007

(30) Foreign Application Priority Data
Mar. 4, 2004 (IT) MO2004A000050

(51) Int. Cl. A61B 6/00

(52) U.S. Cl. 600/473

(57) ABSTRACT

A method for generating and processing images for use in dentistry comprising the steps of. acquiring the position of at least one first reference surface, associated with at least one portion of either the upper dental arch or the lower dental arch of a patient, and of at least one second reference surface, which is associated with at least one portion of the other of the dental arches and/or with a handpiece operated by a health operator inside the mouth of the patient; transducing the acquired positions of the first and second surfaces into signals to be transmitted to an electronic processing unit, in which CAD software is implemented; processing, with the processing unit, the transmitted signals in order to generate an image for use in dentistry; and displaying the image on a screen.

### TECHNICAL FIELD

The present invention relates to a method for generating and processing images for use in dentistry.

### BACKGROUND ART

In dentistry, the need is known to acquire static and possibly dynamic images of the teeth of patients in order to reproduce or improve their structural and aesthetic configuration following conservative procedures, procedures for prosthetic reconstruction, or prosthodontics, or to study their occlusion or the kinematics/dynamics of mastication or to assist in the production of dental prostheses, such as crowns, bridges, inlays or onlays.

With particular reference to the production of prostheses, it is known that dentists entrust their manufacture to external dental-technician services, which are collaborators/suppliers thereof.

The known method of lost-wax casting is used to manufacture dental prostheses.

This method substantially consists in obtaining a wax model of the prosthesis by using a plaster cast, obtained by pouring plaster into an impression of the dental arch to be reconstructed.

The wax model is then covered with a layer of refractory material, which is heated in a furnace after it has hardened; the wax bums, leaving a casting mold made of refractory material.

The material that constitutes the prosthesis, for example a molten metal, is poured into the mold, and after said material has hardened the mold is broken to extract the resulting prosthesis.

The prosthesis is then subjected to finishing treatments and finally covered with a ceramic cladding material and subjected to an additional thermal hardening treatment.

This known method is disadvantageously long and laborious, consists of many complicated steps, and requires many manual operations on the part of the dental technician.

Moreover, it does not ensure the production of prostheses of constant high quality, since it depends on the skill and experience of the dental technician.

Nonetheless, reconstructing teeth with this method requires many patient sessions, during which the dentist prepares the tooth as a stump, takes the impression to be supplied to the dental technician, tests the prosthesis being manufactured, et cetera; each session can last even more than one hour, and the time between one session and the next can vary from a few days to a few weeks, with consequent inconvenience for patients and dentists.

Finally, it should be noted that a temporary prosthesis is applied to the patient from when the stump is prepared until the final prosthesis is applied; in each session, the temporary prosthesis has to be removed and reapplied with the aid of appropriate adhesives (cements), and every time it is necessary to clean adhesive residues off the site where the prosthesis is applied.

Methods for producing dental prostheses that use systems of the CAD-CAM (Computer-Aided Design and Manufacturing) type are also known.

These last methods substantially consists in acquiring the image of the prosthesis application site and possibly of the teeth that are adjacent and/or antagonist thereto; in feeding said image to a computer; in designing, by using the acquired image and any additional data provided in input, the prosthesis to be manufactured; in processing said design so as to obtain from it data for controlling and actuating a milling unit; and in transmitting the resulting data to said milling unit in order to actuate and control its tool, which by working on a block of raw material (metal or ceramic) obtains the designed prosthesis from it.

The image of the prosthesis application site is acquired either by digital photography or by means of the electronic scanning of a plaster model obtained by means of an impression made with polymeric material of the dental arch in which it is provided.

With respect to the lost-wax casting method, methods that use systems of the CAD-CAM type are quicker and less laborious and allow to obtain prostheses of constant and high quality that have an accurate shape.

However, even these known methods, which use CAD-CAM systems, are not free from drawbacks, including the fact that particularly if the image of the prosthesis application site is obtained from a model of the dental arch in which it is formed, they require laborious and time-consuming operations for forming the impression and the model and for scanning said model, and the fact that if the image of the application site is obtained by means of a digital camera they do not allow to acquire extensive and complete images.

Another drawback of known methods is that they do not allow to produce prostheses consisting of more than two or three consecutive teeth.

Another drawback of known methods is that they are limited to the production of prostheses and provide no visual aid in preparing the application sites of said prostheses or in studying the teeth (in terms of aesthetics and occlusion) of the patients as a preliminary to any dental procedure.

### DISCLOSURE OF THE INVENTION

The aim of the present invention is to eliminate the above-mentioned drawbacks, by providing a method for generating and processing images for use in dentistry that allows to acquire static and dynamic images simply and rapidly without the aid of impressions.

Within this aim, an object of the present invention is to provide a method for generating and processing images-for manufacturing prostheses, including prostheses consisting of more than three consecutive teeth, that are integrated with the teeth of the patients in terms of aesthetics and occlusion.

Another object of the present invention is to provide a method that assists in the study of the teeth of patients in terms of aesthetics and occlusion, as a preliminary to a generic dental procedure and/or in preparing prosthesis application sites.

Another object of the present invention is to provide a method that is simple, relatively easy to provide in practice, safe in use, effective in operation, and has a relatively low cost.

This aim and these and other objects that will become better apparent hereinafter are achieved by the present method for generating and processing images for use in dentistry, characterized in that it comprises: acquiring the position of at least one first reference surface, associated with at least one portion of either the upper dental arch or the lower dental arch of a patient, and of at least one second reference surface, which is associated with at least one portion of the other of said upper and lower dental arches and/or with a handpiece operated by a health operator inside the mouth of the patient; transducing the acquired positions of said first surface and of said second surface into signals to be transmitted to an electronic processing unit, in which CAD software is implemented; processing, with said processing unit, the transmitted signals in order to generate an image for use in dentistry; and displaying said image on a screen.

### BRIEF DESCRIPTION OF THE DRAWINGS

Further characteristics and advantages of the present invention will become better apparent from the following detailed description of a preferred but not exclusive embodiment of a method for generating and processing images for use in dentistry, illustrated by way of non-limiting example in the accompanying drawings, wherein:

FIG. 1 is a block diagram of the method according to the invention;

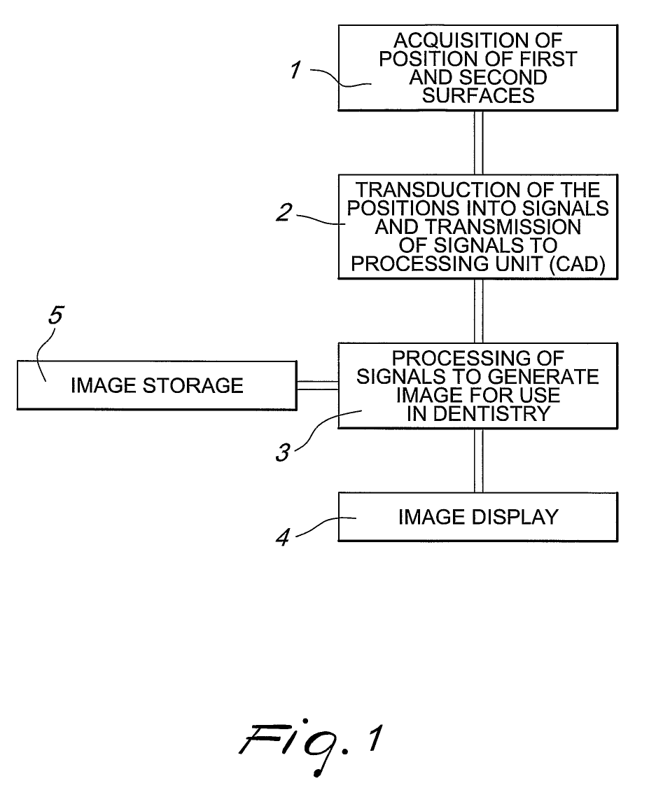

FIG. 2 is a block diagram of an alternative embodiment of the method according to the invention.

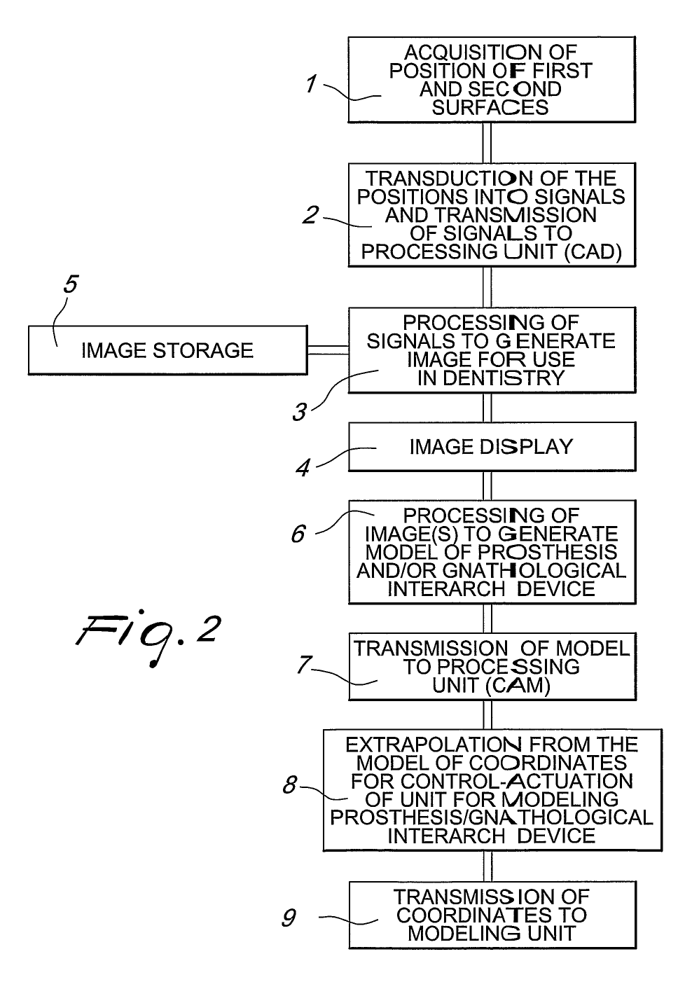

### WAYS OF CARRYING OUT THE INVENTION

With reference to the figures, the method according to the invention comprises: a step 1 for acquiring the position of at least one first reference surface, which is associated with at least one portion of either the upper dental arch or the lower dental arch of a patient, and of at least one second reference surface, which is associated with at least one portion of the other of said upper and lower dental arches and/or with a handpiece that is operated by a health operator inside the mouth of the patient; a step 2 for transducing the acquired positions of the first and second surfaces into signals and for transmitting said signals to an electronic processing unit, in which CAD software is implemented; a step 3 for processing, by means of the electronic processing unit, the signals transmitted thereto in order to generate an image for use in dentistry; and a step 4 for displaying the generated image on a screen.

The method may further comprise a step 5 for storing the generated image in a memory unit associated with the processing unit.

In the present description, the terms “upper dental arch” and “lower dental arch” designate the series of teeth or osteointegrated implants inserted in the alveoles of the bone of, respectively, the maxilla and the mandible; the term “prosthesis” designates an artificial device that replaces a portion of a tooth, an entire tooth, or a plurality of adjacent teeth, such as for example inlays, outlays, crowns or bridges; the expression “prosthesis application site” designates the location where said prosthesis is applied, which can be constituted for example by a stump (a natural one, i.e., obtained from a tooth, or an artificial one, i.e., applied to an osteointegrated implant); and the expression “gnathological interarch device” designates a device for correcting the occlusion of the mandible and/or maxilla, such as for example so-called “bites” made of resin.

As will become better apparent in the continuation of the present description, as the position of the first and second surfaces varies, the processed image may be of various kinds and may represent various features, such as for example:
the occlusion relationship of the mandible and maxilla,
the kinematics/dynamics of mastication,
the shape of one or both of the upper and lower dental arches or of a portion thereof before a dental procedure,
the evolution of the shape of a site of application of a prosthesis during its preparation and/or final shaping.

Moreover, the uses of these images may be various: for example, they can be used, individually or in combination with each other, in order to:
study the occlusion of the maxilla and mandible and the kinematics/dynamics of mastication,
acquire and/or analyze, from the structural and aesthetic standpoint, the current state of the teeth as a preliminary to a procedure,
assist the health operator in a procedure, such as for example the preparation of an application site of a prosthesis,
creating a model of the prosthesis or of a gnathological interarch device for their production, and others.

If, for example, the first surface is associated with at least one portion of either the upper dental arch or the lower dental arch and the second surface is associated with at least one portion of the other one of said arches, it is possible to obtain an image, for example in the form of a three-dimensional diagram, that represents the relative motion between the maxilla and the mandible and can be used for gnathological analyses of occlusion and/or of the kinematics/dynamics of mastication and/or to produce a gnathological interarch device and/or to integrate the generation of prostheses with a correct occlusal adjustment.

If instead the first surface is associated with at least one portion of either the upper dental arch or the lower dental arch and the second surface is associated with a handpiece of the probe type, it is possible to obtain a three-dimensional image of the state, i.e., of the structural configuration and/or aesthetic appearance, of said portion prior to a dental procedure, for example a reconstruction by prosthesis, a conservative procedure, a prosthodontic procedure, or others.

If instead the first surface is associated with at least one portion of either the upper dental arch or the lower dental arch in which a prosthesis application site is being formed or is already formed, while the second surface is associated with a handpiece, of the tool and/or probe type, used respectively in the final step for preparing said site or to probe the prepared application site, it is possible to obtain a three-dimensional image of said site, which can be used for example to generate a model for manufacturing the prosthesis to be applied therein.

Finally, if the first surface is associated with at least one portion of either the upper dental arch or the lower dental arch where it is necessary to define a prosthesis application site and the second surface is associated with a handpiece of the tool type for preparing said site, it is possible to obtain, during the preparation of said site, a three-dimensional image of the relative position of the handpiece with respect to the affected upper or lower arch portion, for assisted preparation of said application site.

In this last case, the method according to the invention can comprise the definition of a first axis of reference of the upper or lower dental arch portion in which it is necessary to form the application site and of a second reference axis of the handpiece; the acquisition of the relative position of the second axis with respect to the first axis; the comparison between the acquired relative position and predefined limit reference positions stored in the electronic processing unit; and the indication of any straying of the acquired relative position beyond the limit reference positions.

The relative position of the first and second axes can be defined by their angle of incidence, and the limit reference positions can be defined by the maximum or minimum breadth of said angle of incidence.

During the assisted preparation of an application site, the method according to the invention can comprise the control of the amount of material removed by the tool handpiece from the tooth or from the osteointegrated implant being worked and/or of the height thereof, in order to prevent respectively the former from exceeding a predefined maximum value and the latter from dropping below a predefined minimum value.

The acquisition step 1 can be of the so-called active or passive type; in the first case, it comprises the emission of electromagnetic radiation on the part of the first and second surfaces and the reception of the radiation emitted thereby; in the second case, it comprises the emission of electromagnetic radiation toward the first and second surfaces and the reception of the radiation reflected/absorbed thereby.

The electromagnetic radiation preferably belongs to the infrared range.

In an alternative embodiment, the method according to the invention also comprises a step 6 for processing at least the image of an application site and/or the image that represents the relative motion between the maxilla and the mandible in order to generate, by CAD, the three-dimensional model respectively of the prosthesis to be applied to said site and/or of a gnathological interarch device for correcting, for example, defects in the occlusion of the mandible and/or maxilla.

If the processed image is the image of a prosthesis application site in order to generate the model of said prosthesis, said image can be processed in combination with at least one image selected from the group that comprises:
the image of the portions of the upper dental arch or of the lower dental arch that are adjacent thereto;
the image of the portions of the upper dental arch or lower dental arch that are antagonist thereto;
the image of the state of the portion of the upper dental arch or lower dental arch in which it is formed, before the creation of said site;
the image, for example in the form of a three-dimensions chart, that represents the relative motion between the maxilla and the mandible;
the image or images of a database of models of teeth, or others;

all acquired and/or processed and stored beforehand.

In this manner, it is possible to create the three-dimensional model of a prosthesis whose shape is suitable to reproduce or modify the occlusal relationship with the dental arch portions that are adjacent and/or antagonist to the site and/or reproduce or modify the structural and/or aesthetic state of the portion of the upper dental arch or lower dental arch in which the application site is formed prior to the creation of said site.

It is thus possible to create prostheses that reproduce the original teeth or modify and improve them.

Finally, the method comprises: a step 7 for transmitting the model of the prosthesis or of the gnathological interarch device obtained in the processing step 6 to an auxiliary electronic processing unit in which CAM software is implemented; a step 8 for extrapolating from the transmitted model coordinates for controlling and actuating an electronically-controlled modeling unit; and a step 9 for transmitting the extrapolate d coordinates to the modeling unit in order to manufacture the prosthesis or the gnathological interarch device that corresponds to the model generated in the processing step 6.

Conveniently, the electronic processing unit and the auxiliary electronic processing unit coincide with each other.

The modeling unit is generally constituted by a unit for milling a block of material and can be of a remote type.

In practice it has been found that the described invention achieves the intended aim and objects.

The invention thus conceived is susceptible of numerous modifications and variations, all of which are within the scope of the appended claims.

All the details may further be replaced with other technically equivalent ones.

In practice, the materials used, as well as the shapes and the dimensions, may be any according to requirements without thereby abandoning the scope of the protection of the appended claims.

The disclosures in Italian Patent Application No. MO2004A000050 from which this application claims priority are incorporated herein by reference.

1. A method for generating and processing images for use in dentistry, characterized in that it comprises the steps of: acquiring the position of at least one first reference surface, associated with at least one portion of either the upper dental arch or the lower dental arch of a patient, and of at least one second reference surface, which is associated with at least one portion of the other of said upper and lower dental arches and/or with a handpiece operated by a health operator inside the mouth of the patient; transducing the acquired positions of said first surface and of said second surface into signals to be transmitted to an electronic processing unit, in which CAD software is implemented; processing, with said processing unit, the transmitted signals in order to generate an image for use in dentistry; and displaying said image on a screen.

2. The method according to claim 1, characterized in that said acquisition step comprises the emission of electromagnetic radiation from said first and second surfaces and the reception of the emitted radiation.

3. The method according to claim 1, characterized in that said acquisition step comprises the emission of electromagnetic radiation toward said first and second surfaces and the reception of the radiation reflected/absorbed by said first and second surfaces.

4. The method according to one or more of the preceding claims, characterized in that said first surface is associated with at least one portion of either said upper dental arch or said lower dental arch and said second surface is associated with at least one portion of the other of said upper and lower dental arches, in order to generate an image that represents the relative motion between said maxilla and said mandible for gnathological analyses of occlusion and/or of the kinematics/dynamics of mastication and/or for generating a gnathological interarch device and/or for integrating the creation of prostheses with correct occlusal adjustment.

5. The method according to one or more of the preceding claims, characterized in that said first surface is associated with at least one portion of either said upper dental arch or said lower dental arch and said second surface is associated with a handpiece of the probe type in order to create a three-dimensional image of the state of said portion before a dental procedure.

6. The method according to one or more of the preceding claims, characterized in that said first surface is associated with at least one portion of either said upper dental arch or said lower dental arch and said second surface is associated with a handpiece, of the tool and/or probe type, for generating a three-dimensional image of the application site of a prosthesis, formed in said portion, in order to produce said prosthesis.

7. The method according to one or more of the preceding claims, characterized in that said first surface is associated with at least one portion of either said upper dental arch or said lower dental arch and said second surface is associated with a handpiece of the tool type for generating a three-dimensional image of the relative position of said handpiece with respect to said portion for the assisted preparation of said application site.

8. The method according to claim 7, characterized in that it comprises the steps of: defining a first reference axis in said portion for preparing said application site and a second reference axis of said handpiece; acquiring the relative position of said second axis with respect to said first axis; comparing the acquired relative position with predefined limit reference positions stored in said electronic processing unit; and indicating the straying of said acquired relative position beyond said limit reference positions.

9. The method according to claim 8, characterized in that said relative position is defined by the angle of incidence formed by said first and second axes, said limit reference positions being defined by the maximum or minimum breadth of said angle of incidence.

10. The method according to one or more of claims 7 to 9, characterized in that it comprises detecting the amount of material removed by the tool handpiece from the tooth or osteointegrated implant being worked in said portion and/or the height thereof; comparing said acquired quantity and/or height respectively with a predefinable maximum reference value and with a predefinable minimum reference value; and indicating the straying of said acquired quantity and/or height beyond said maximum and minimum reference values.

11. The method according to one or more of the preceding claims, characterized in that said electromagnetic radiation belongs to the infrared range.

12. The method according to one or more of the preceding claims, characterized in that it comprises storing said images in a memory unit associated with said processing unit.

13. The method according to one or more of the preceding claims, characterized in that it comprises processing at least the image of said application site and/or the image that represents the relative motion between said maxilla and said mandible in order to generate, with said CAD, the three-dimensional model respectively of said prosthesis and/or of a gnathological interarch device.

14. The method according to one or more of the preceding claims, characterized in that it comprises processing the image of said application site and of at least one image selected from the group that comprises:
the image of the portions of the upper dental arch or of the lower dental arch that are adjacent thereto;
the image of the portions of the upper dental arch or lower dental arch that are antagonist thereto;
the image of the state of the portion of the upper dental arch or lower dental arch in which it is formed, before the creation of said site;
the image that represents the relative motion between the maxilla and the mandible;
the image or images of a database of models of teeth, or others; in order to create, with said CAD, the three-dimensional model of said prosthesis with a shape suitable to reproduce or modify the occlusal relationship with said adjacent and/or antagonist portions and/or reproduce or modify the state of the portion of the upper or lower dental arch in which said application site is formed prior to the creation of said site.

15. The method according to one or more of the preceding claims, characterized in that it comprises the steps of: transmitting said model of said prosthesis or of said gnathological prosthesis to an auxiliary electronic processing unit in which CAM software is implemented; extrapolating from said model the coordinates for controlling and actuating an electronically-controlled modeling unit; and transmitting said coordinates to said modeling unit in order to manufacture said prosthesis or said gnathological prosthesis.

16. The method according to one or more of the preceding claims, characterized in that said electronic processing unit and said auxiliary electronic processing unit mutually coincide.

17. The method according to one or more of the preceding claims, characterized in that said modeling unit is a unit for milling a block of material.
